(19) United States
(12) Patent Application Publication
(10) Pub. No.: US 2021/0310053 A1
(43) Pub. Date: Oct. 7, 2021

(54) METHODS AND SYSTEMS FOR TARGET SCREENING

(71) Applicant: ThinkCyte, Inc., Tokyo (JP)

(72) Inventor: Keiki Sugimoto, Tokyo (JP)

(21) Appl. No.: 17/231,725

(22) Filed: Apr. 15, 2021

Related U.S. Application Data
(63) Continuation of application No. PCT/US2019/056743, filed on Oct. 17, 2019.
(60) Provisional application No. 62/825,524, filed on Mar. 28, 2019.
(60) Provisional application No. 62/747,620, filed on Oct. 18, 2018.

(51) Int. Cl. C12Q 1/6809; G16B 40/20

(52) U.S. Cl. CPC C12Q 1/6809; G16B 40/20

(57) ABSTRACT

The present disclosure provides a method for identifying a nucleic acid, which may comprise incubating a cell that has been or is suspected of having been transfected or transduced with an exogenous ribonucleic acid (RNA) molecule or an exogenous deoxyribonucleic (DNA) molecule. Next, a morphological change of the cell may be identified. Next, contents of the cell may be processed to identify a nucleic acid sequence or a peptide, polypeptide, or protein or a sequence of the peptide, polypeptide, or protein. Next, the nucleic acid sequence or the peptide, polypeptide, or protein or the sequence of the peptide, polypeptide, or protein may be analyzed to determine an exogenous sequence of the exogenous RNA molecule or the exogenous DNA molecule. Next, the exogenous sequence of the exogenous RNA molecule or the exogenous DNA molecule may be identified as effecting the morphological change of the cell. The exogenous RNA molecule or the exogenous DNA molecule may encode genes or peptides, polypeptides, or proteins that inhibit, activate, or modulate a biochemical pathway within the cell, thereby causing the morphological change of the cell.

### CROSS-REFERENCE

This application is a continuation application of International Application No. PCT/US2019/056743, filed Oct. 17, 2019, which claims the benefit of U.S. Provisional Patent Application No. 62/747,620, filed on Oct. 18, 2018, and U.S. Provisional Patent Application No. 62/825,524, filed on Mar. 28, 2019, each of which is entirely incorporated by reference for all purposes.

### BACKGROUND

Pooled genetic screening is a very powerful tool to identify the genes responsible for pathways of interest. So far, selection methods for pooled genetic screening mostly rely on positive screening, negative screening, or fluorescence intensity-based screening by fluorescence-activated cell sorting (FACS).

Image-based screening is also a very useful screening method to identify genes that may have an important role in the pathways of interest. Most image-based screening methods can only be performed by microscopes and may not be applied to a pooled format screening.

### SUMMARY

Presented herein are systems and methods for rapid image-based pooled genetic/peptide screening (RIPGS). Such systems and methods may represent a new and high-throughput hit identification platform for undruggable targets such as transcription factors, RAS and phosphatase. Also, RIPGS may be applied to identify one or more genes that are responsible for nuclear translocation of transcription factors, protein aggregation, organelle dysfunction, or the like. One or more peptide expression libraries, ribonucleic acid (RNA) libraries, short hairpin RNA (shRNA) libraries, small interfering RNA (siRNA) libraries, micro RNA (miRNA) libraries, antisense RNA (asRNA) libraries, or clustered regularly interspersed palindromic repeat (CRISPR) guide RNA (gRNA) libraries may be transduced in one or more cells. A guide RNA library may be a single guide RNA (sgRNA) library. Once cells have been transfected or transduced using the libraries and are ready to analyze, cells of interest may be sorted based on fluorescence signal patterns that display behavior such as protein localization. The sorted cells may be analyzed by nucleic acid sequencing, such as next-generation sequencing (NGS), to determine which plasmids were transfected or transduced in the cells. This first in class drug discovery platform will benefit not only industries but also patients.

In various aspects, the present disclosure provides a method for identifying a nucleic acid molecule, comprising: (a) providing a cell that has been or is suspected of having been transfected or transduced with at least one exogenous ribonucleic acid (RNA) molecule or at least one exogenous deoxyribonucleic (DNA) molecule, wherein said cell is among a population of cells; (b) subsequent to (a), identifying a morphological change of said cell; (c) processing a content(s) of said cell to identify (i) a nucleic acid sequence or (ii) a peptide, polypeptide, or protein or (iii) a sequence of said peptide, polypeptide, or protein; (d) analyzing (i) said nucleic acid sequence or (ii) said peptide, polypeptide, or protein or (iii) said sequence of said peptide, polypeptide, or protein to determine said exogenous sequence of said exogenous RNA molecule or said exogenous DNA molecule; and (e) identifying an exogenous sequence of said exogenous RNA molecule or said exogenous DNA molecule as effecting said morphological change of said cell.

In some aspects, said exogenous sequence of said exogenous RNA molecule or said exogenous DNA molecule is unknown. In some aspects, said exogenous sequence of said exogenous RNA molecule or said exogenous DNA molecule is known. In some aspects, said exogenous RNA molecule or said exogenous DNA molecule encodes a gene, peptide, polypeptide, or protein.

In some aspects, said morphological change comprises one or more members selected from the group consisting of: a change in a protein-protein interaction within said cell, a change in protein localization within said cell, a change in shape of said cell, a change in shape of one or more components of said cell, and a change in shape of one or more organelles of said cell. In some aspects, said cell is transfected using a plasmid comprising said exogenous RNA molecule or said exogenous DNA molecule.

In some aspects, (c) comprises sequencing at least one nucleic acid molecule or at least one protein molecule from said cell. In some aspects, said sequencing comprises massively parallel array sequencing. In some aspects, said sequencing comprises sequencing by synthesis. In some aspects, said sequencing comprises next generation sequencing.

In some aspects, said morphological change is identified using optical microscopy. In some aspects, said optical microscopy comprises confocal microscopy.

In some aspects, in (b), said morphological change of said cell is identified while said cell is flowing in a flow cell or flow channel. In some aspects, said morphological change is identified in (b), said cell is not immobilized or fixed. In some aspects, when said morphological change is identified in (b), said cell is fixed.

In some aspects, said population of cells is heterogeneous. In some aspects, (b) is repeated for each cell of a plurality of cells among said population of cells. In some aspects, (b) is repeated at a rate of at least about 1500 cells per second (cells/s). In some aspects, (b) is repeated at a rate of at least about 2000 cells/s, at least about 3000 cells/s, at least about 4000 cells/s, at least about 5000 cells/s, at least about 6000 cells/s, at least about 7000 cells/s, at least about 8000 cells/s, at least about 9000 cells/s, or at least about 10,000 cells/s.

In some aspects, said cell is isolated from said population of cells based on said morphological change. In some aspects, said population of cells is not immobilized or fixed. In some aspects, said population of cells is fixed. In some aspects, said morphological change is identified in (b), said population of cells is flowing through a flow cell or flow channel. In some aspects, each cell among said population of cells comprises at least one randomly-inserted exogenous RNA molecule or at least one randomly-inserted exogenous DNA molecule.

In some aspects, (b) comprises imaging said cell. In some aspects, (b) comprises obtaining temporal signals containing image information. In some aspects, said temporal signals are transformed into time-independent image information. In some aspects, (b) comprises obtaining temporal signals containing spatial information. In some aspects, said temporal signals are transformed into time-independent spatial information.

In some aspects, the method further comprises isolating said cell upon identifying said morphological change within said cell, and subsequently obtaining said sequencing information from said cell. In some aspects, said cell is isolated from a plurality of cells.

The method of claim 1, further comprising using at least said morphological change to determine that said exogenous DNA molecule or said RNA molecule inhibits or activates a biochemical pathway within said cell.

In some aspects, (b) comprises using a machine learning algorithm to identify said morphological change. In some aspects, said machine learning algorithm comprises one or more members selected from the group consisting of: support vector machines, random forest, artificial neural networks, convolutional neural networks, deep learning, ultra-deep learning, gradient boosting, AdaBoosting, decision trees, linear regression, and logistic regression. In some aspects, said machine learning algorithm is trained using a training set comprising image information of a cell population positive for said morphological change and image information of a cell population negative for said morphological change. In some aspects, said machine learning algorithm is trained using a training set comprising spatial information of a cell population positive for said morphological change and spatial information of a cell population negative for said morphological change.

In some aspects, the method further comprises, prior to (b), labeling said peptides, polypeptides, or proteins within said cell. In some aspects, said labeling comprises labeling said one or more peptides, polypeptides, or proteins with one or more fluorescent labels, Forster resonance energy transfer (FRET) labels, dyes, fluorophores, or quantum dots.

In various aspects, the present disclosure provides a method for screening a cell for a target nucleic acid, peptide, polypeptide, or protein, comprising (a) providing a cell that has been or is suspected of having been transfected or transduced with an exogenous ribonucleic acid (RNA) molecule or an exogenous deoxyribonucleic (DNA) molecule, wherein said cell is among a population of cells, and wherein said exogenous RNA or DNA causes said cell to express a population of macromolecules within said cell, (b) identifying a morphological change of said cell in response to said population of macromolecules expressed by said cell, (c) subjecting a genome of said cell to sequence identification to identify said exogenous RNA or DNA, and (d) determining that said exogenous RNA or DNA induced said morphological change.

In some aspects, identifying said morphological change comprises identifying a localization profile or pattern of macromolecules of said population of macromolecules. In some aspects, identifying said morphological change comprises identifying a shape of said cell or one or more sub-cellular constituents of said cell. In some aspects, said one or more sub-cellular constituents comprise an organelle of said cell. In some aspects, said population of macromolecules comprises a population of one or more members selected from the group consisting of peptides, polypeptides, proteins and nucleic acid molecules.

In various aspects, the present disclosure provides a system for identifying a nucleic acid molecule, comprising one or more computer processors that are individually or collectively programmed to: (a) identify a morphological change of a cell, which cell has been or is suspected of having been transfected or transduced with at least one exogenous ribonucleic acid (RNA) molecule or at least one exogenous deoxyribonucleic (DNA) molecule, wherein said cell is among a population of cells; (b) process a content(s) of said cell to identify (i) a nucleic acid sequence or (ii) a peptide, polypeptide, or protein or a sequence of said peptide, polypeptide, or protein; (c) analyze (i) said nucleic acid sequence or (ii) said peptide, polypeptide, or protein or said sequence of said peptide, polypeptide, or protein to determine said exogenous sequence of said exogenous RNA molecule or said exogenous DNA molecule; and (d) identify an exogenous sequence of said exogenous RNA molecule or said exogenous DNA molecule as effecting said morphological change of said cell.

In various aspects, the present disclosure provides a system for screening a cell for a target nucleic acid, peptide, polypeptide, or protein, comprising one or more computer processors that are individually or collectively programmed to: (a) identify a morphological change of a cell in response to a population of macromolecules expressed by said cell, wherein said cell has been or suspected of having been transfected or transduced with an exogenous ribonucleic acid (RNA) molecule or an exogenous deoxyribonucleic (DNA) molecule, wherein said cell is among a population of cells, and wherein said exogenous RNA or DNA causes said cell to express said population of macromolecules within said cell; (b) subject a genome of said cell to sequence identification to identify said exogenous RNA or DNA; and (c) determine that said exogenous RNA or DNA induced said morphological change.

An aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a computer system that is programmed or otherwise configured to implement methods provided herein.

FIG. 2A shows an example of a method for pooled genetic screening.

FIG. 2B shows an example of a method for image-based genetic screening.

FIG. 3 shows an example of a method for image-based pooled genetic screening.

FIG. 4A shows fluorescence images of cells with fluorescently labeled mitochondria and cells with fluorescently labeled lysosomes.

FIG. 4B shows classification of cells with fluorescently labeled mitochondria and cells with fluorescently labeled lysosomes using systems and methods of the present disclosure.

FIG. 4C shows fluorescence images of STAT3 proteins localized within a nucleus (nuclear localized) and outside of a nucleus (cytoplasmic).

FIG. 4D shows classification of STAT3 proteins as localized within a nucleus or outside of a nucleus (cytoplasm) using systems and methods of the present disclosure.

FIG. 4E shows fluorescence images of unaggregated proteins and aggregated protein complexes in a cell.

FIG. 4F shows classification of proteins as unaggregated proteins or aggregated protein complexes using systems and methods of the present disclosure.

FIG. 4G shows fluorescence images of cells with nuclear-localized nuclear factor kappa-light-chain-enhancer of activated B cells (NF-κB) proteins and cells with cytoplasmic NF-κB proteins.

FIG. 4H shows classification of cells with nuclear-localized NF-κB proteins and cells with cytoplasmic NF-κB proteins using systems and methods of the present disclosure.

FIG. 5 shows a flowchart for an example of a method for identifying a nucleic acid molecule.

FIG. 6 shows an example of a method for identifying a nucleic acid molecule or a peptide, polypeptide, or protein based on protein-protein interactions, protein aggregation, or protein localization.

FIG. 7 shows an example of a method for target discovery using image-based pooled genetic screening.

FIG. 8A shows fluorescence images of cells with nuclear-localized p65 proteins and cells with cytoplasmic p65 proteins.

FIG. 8B shows classification of cells with nuclear-localized p65 proteins and cells with cytoplasmic p65 proteins using systems and methods of the present disclosure.

FIG. 9A shows classification of cells with nuclear-localized fluorescent proteins and cells with cytoplasmic fluorescent proteins using an image-based system prior to sorting.

FIG. 9B shows classification of cells with nuclear-localized fluorescent proteins and cells with cytoplasmic fluorescent proteins using an image-based system following sorting using systems and methods of the present disclosure.

FIG. 10A shows fluorescence images of cells with nuclear-localized nuclear factor kappa-light-chain-enhancer of activated B cells (NF-κB) proteins and cells with cytoplasmic NF-κB proteins.

FIG. 10B shows classification of cells with nuclear-localized NF-κB proteins and cells with cytoplasmic NF-κB proteins using systems and methods of the present disclosure.

FIG. 10C shows fluorescence images of cells with p53 bound to MDM2 and cells with p53 not bound to MDM2.

FIG. 10D shows classification of cells with p53 bound to MDM2 and cells with p53 not bound to MDM2 using systems and methods of the present disclosure.

FIG. 10E shows fluorescence images of cells with fluorescently labeled mitochondria and cells with fluorescently labeled lysosomes.

FIG. 10F shows classification of cells with fluorescently labeled mitochondria and cells with fluorescently labeled lysosomes using systems and methods of the present disclosure.

FIG. 11 shows sorting of cells with nuclear-localized NF-κB or cytoplasmic NF-κB using systems and methods of the present disclosure and subsequent verification using an image-based system and fluorescence-activated cell sorting (FACS).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

In an aspect, the present disclosure provides a method for identifying a nucleic acid molecule, which may comprise providing a cell that has been or is suspected of having been transfected or transduced with at least one exogenous ribonucleic acid (RNA) molecule or at least one exogenous deoxyribonucleic (DNA) molecule. The nucleic acid molecule may confer a property, e.g., a morphological change, on a cell. Next, a morphological change of the cell may be identified. Next, contents of the cell may be processed to identify (i) a nucleic acid sequence or (ii) a peptide, polypeptide, or protein or a sequence of the peptide, polypeptide, or protein. Next, (i) the nucleic acid sequence or (ii) the peptide, polypeptide, or protein or the sequence of the peptide, polypeptide, or protein may be analyzed to determine the exogenous sequence of the exogenous RNA molecule or the exogenous DNA molecule. Next, the exogenous sequence of the exogenous RNA molecule or the exogenous DNA molecule may be identified as effecting the morphological change of the cell.

The exogenous sequence of the exogenous RNA molecule or the exogenous DNA molecule may be unknown. The exogenous sequence of the exogenous RNA molecule or the exogenous DNA molecule may be known. The exogenous sequence of the exogenous RNA molecule or the exogenous DNA molecule may encode a gene, peptide, polypeptide, or protein.

The morphological change may comprise one or more members selected from the group consisting of: a change in a protein-protein interaction within the cell, a change in protein localization within the cell, a change in shape of the cell, a change in shape of one or more components of the cell, and a change in shape of one or more organelles of the cell.

The morphological change of the cell may be effected directly or indirectly by the exogenous RNA molecule or the exogenous DNA molecule. In some cases, the exogenous RNA molecule or the exogenous DNA molecule may directly effect the morphological change by directly interacting with one or more members selected from the group consisting of: a protein, a small molecule, an ion, a cofactor, an organelle, a phospholipid, or a nucleic acid. In some cases, the exogenous RNA molecule or the exogenous DNA molecule may indirectly effect the morphological change by encoding a protein or nucleic acid that directly effects the morphological change. The protein or nucleic acid that directly effects the morphological change may effect the morphological change by directly interacting with one or more members selected from the group consisting of: a protein, a small molecule, an ion, a cofactor, an organelle, a phospholipid, or a nucleic acid. In some cases, the exogenous RNA molecule or the exogenous DNA molecule may indirectly effect the morphological change by encoding a protein or nucleic acid that indirectly effects the morphological change. The protein or nucleic acid that indirectly effects the morphological change may effect the morphological change indirectly by interacting with or altering the interaction of one or more members selected from the group consisting of: a protein, a small molecule, an ion, a cofactor, an organelle, a phospholipid, or a nucleic acid, wherein altering the interaction effects a morphological change downstream of the interaction.

In some cases, imaging may be performed using the methods and systems (referred to herein as “ghost cytometry”) disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference.

The cell may be transfected or transduced using a plasmid comprising the exogenous RNA molecule or the exogenous DNA molecule.

Sequencing information may be obtained from the cell by sequencing at least one DNA molecule and/or at least one RNA molecule from the cell (e.g., transduced exogenous RNA or DNA molecule, a genome and/or transcriptome of the cell).

The sequencing may be massively parallel array sequencing. The sequencing may comprise sequencing by synthesis (e.g., Illumina, Pacific Biosciences of California or Ion Torrent). The sequencing may be single molecule sequencing (e.g., Oxford Nanopore). The sequencing may comprise next generation sequencing.

In some cases, the exogenous RNA molecule or exogenous DNA molecule is designed to encode peptides, polypeptides, or proteins that have a stable α-helical structure, which may be a critical aspect of peptide-protein interactions. The peptide encoding RNA or DNA may be cloned to plasmid vectors for transfection or transduction into a cell. The cell may express the encoded peptide, polypeptide, or protein.

In some cases, the exogenous RNA molecule or exogenous DNA molecule is transfected into the cell using clustered regularly interspaced short palindromic repeats (CRISPR), such as CRISPR-associated 9 (Cas9), Cas13d, CasX or other variants, or other gene editing technologies, such as Zinc-finger nucleases (ZFN) or transcription activator-like effector nuclease (TALEN), as described in Shalem O., Sanjana N E., Hartenian E., Shi X., Scott D A., Mikkelson T., Heckl D., Ebert B L., Root D E., Doench J G., Zhang F., Science. 2014, 343 (6166):84-87. doi: 10.1126/science.1247005. Genome-scale CRISPR-Cas9 knockout screening in human cells; and Joung J., Konermann S., Gootenberg J S., Abudayyeh O O., Platt R J., Brigham M D., Sanjana N E., Zhang F., Nat Protoc. 2017, 12(4):828-863. doi:10.1038/nprot.2017.016. Genome-scale CRISPR-Cas9 knockout and transcriptional activation screening, each of which is entirely incorporated herein by reference.

In some cases, a genome scale CRISPR library or peptide expression library is amplified.

In some instances, the morphological change is identified using optical microscopy. In some instances, the morphological change is identified using fluorescence microscopy. In some instances, the morphological change is identified using confocal microscopy. In some instances, the morphological change is identified using super-resolution microscopy. In some instances, the morphological change is identified while the cell is flowing in a flow cell.

The cell may be not immobilized or fixed when the morphological change is identified. The cell may be immobilized or fixed when the morphological change is identified. The cell may be among a population of cells. The population of cells may be not immobilized or fixed. The population of cells may be immobilized or fixed. The population of cells may be flowing through a flow channel when the morphological change is identified.

Identifying the morphological change may comprise imaging the cell. Identifying the morphological change may comprise obtaining temporal signals containing image information. The temporal signals may be transformed into time-independent image information. Identifying the morphological change may comprise obtaining temporal signals containing structural information of the cells. The temporal signals may be transformed into time-independent structural information of the cells.

The method may further comprise isolating the cell upon identifying the morphological change, and subsequently obtaining the sequencing information from the cell. The cell may be isolated from a plurality of cells.

The method may further comprise using at least the morphological change to determine that the exogenous DNA or the exogenous RNA inhibits, activates, or alters a biochemical pathway within the cell. The biochemical pathway may comprise one or more members selected from the group consisting of: a protein-protein interaction, a protein conformational change, a localization, a translocation, a transport, an endocytosis, an exocytosis, a chemical reaction, an enzymatic reaction, a phosphorylation, a dephosphorylation, a hydrolysis, a hydration, a cleavage, or a fusion.

Identifying the morphological change may comprise using a machine learning algorithm to identify the morphological change. The machine learning algorithm may comprise one or more members selected from the group consisting of: support vector machines, random forest, artificial neural networks, convolutional neural networks, deep learning, ultra-deep learning, gradient boosting, AdaBoosting, decision trees, linear regression, and logistic regression, or any other machine learning algorithm.

The presence or absence of the morphological change may be identified in each of a plurality of cells among the population of cells. The presence or absence of the morphological change may be identified in each cell among the population of cells at a rate of at least about 10 cells per second (cells/s), at least about 50 cells/s, at least about 100 cells/s, at least about 500 cells/s, at least about 1000 cells/s, at least about 1500 cells/s, at least about 2000 cells/s, at least about 3000 cells/s, at least about 4000 cells/s, at least about 5000 cells/s, at least about 6000 cells/s, at least about 7000 cells/s, at least about 8000 cells/s, at least about 9000 cells/s, at least about 10,000cells/s, at least about 11,000 cells/s, at least about 12,000 cells/s, at least about 13,000 cells/s, at least about 14,000 cells/s, at least about 15,000 cells/s, at least about 20,000 cells/s, at least about 25,000 cells/s, at least about 30,000 cells/s, at least about 35,000 cells/s, at least about 40,000 cells/s, at least about 50,000 cells/s, or more. The presence or absence of the morphological change may be identified in each cell among the population of cells at a rate of at most about 50,000 cells/s, at most about 40,000 cells/s, at most about 35,000 cells/s, at most about 30,000 cells/s, at most about 25,000 cells/s, at most about 20,000 cells/s, at most about 15,000 cells/s, at most about 14,000 cells/s, at most about 13,000 cells/s, at most about 12,000 cells/s, at most about 11,000 cells/s, at most about 10,000 cells/s, at most about 9,000 cells/s, at most about 8,000 cells/s, at most about 7,000 cells/s, at most about 6,000 cells/s, at most about 5,000 cells/s, at most about 4,000 cells/s, at most about 3,000 cells/s, at most about 2,000 cells/s, at most about 1,500 cells/s, at most about 1,000 cells/s, at most about 500 cells/s, at most about 100 cells/s, at most about 50 cells/s, at most about 10 cells/s, or lower.

The presence or absence of the morphological change may be identified in each cell among the population of cells at a rate of from 10 cells/s to 500 cells/s, from 100 cells/s to 1000 cells/s, from 500 cells/s to 1500 cells/s, from 1000 cells/s to 2000 cells/s, from 1500 cells/s to 2000 cells/s, from 1500 cells/s to 10,000 cells/s, from 1500 cells/s to 15,000 cells/s, from 1500 cells/s to 20,000 cells/s, from 1500 cells/s to 40,000 cells/s, from 1500 cells/s to 50,000 cells/s, from 2000 cells/s to 10,000 cells/s, from 2000 cells/s to 15,000 cells/s, from 2000 cells/s to 20,000 cells/s, from 2000 cells/s to 40,000 cells/s, from 2000 cells/s to 50,000 cells/s, from 5000 cells/s to 10,000 cells/s, from 5000 cells/s to 15,000 cells/s, from 5000 cells/s to 20,000 cells/s, from 5000 cells/s to 40,000 cells/s, from 5000 cells/s to 50,000 cells/s, from 9000 cells/s to 10,000 cells/s, from 9000 cells/s to 15,000 cells/s, from 9000 cells/s to 20,000 cells/s, from 9000 cells/s to 40,000 cells/s, from 9000 cells/s to 50,000 cells/s, from 10,000 cells/s to 15,000 cells/s, from 10,000 cells/s to 20,000 cells/s, from 10,000 cells/s to 40,000 cells/s, from 10,000 cells/s to 50,000 cells/s, from 20,000 cells/s to 40,000 cells/s, or from 20,000 cells/s to 50,000 cells/s.

The method may further comprise, prior to identifying a morphological change of the cell in response to the population of macromolecules expressed by the cell, labeling one or more peptides, polypeptides, and/or proteins within the cell. The labeling may comprise labeling the one or more peptides, polypeptides, and/or proteins with one or more fluorescent labels, Forster resonance energy transfer (FRET) labels, dyes, fluorophores, or quantum dots.

The labeling may comprise labeling the one or more peptides, polypeptides, and/or proteins with one or more members selected from the group consisting of: SYBR green, SYBR blue, 4′,6-diamidino-2-phenylindole (DAPI), propidium iodine, Hoechst, SYBR gold, ethidium bromide, acridine, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and 9-amino-6-chlor-2-methoxyacridine (ACMA), Hoechst 33258, Hoechst 33342, Hoechst 34580, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, and -45 (blue), SYTO-13, -16, -24, -21, -23, -12, -11, -20, -22, -15, -14, and -25 (green), SYTO-81, -80, -82, -83, -84, and -85 (orange), SYTO-64, -17, -59, -61, -62, -60, and -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), SYBR Green I, SYBR Green II, SYBR Gold, CellTracker Green, 7-aminoactinomycin D (7-AAD), ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-amino-4-methylcoumarin-3-acetic acid (AMCA), BODIPY fluorophores, 8-methoxypyrene-1,3,6-trisulfonic acid trisodium salt, 3,6-disulfonate-4-amino-naphthalimide, phycobiliproteins, Atto 390, 425, 465, 488, 495, 532, 565, 594, 633, 647, 647N, 665, 680, and 700 dyes, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BH1-0, BHQ-1, BHQ-3, and BHQ-10, QSY Dye fluorescent quenchers (from Molecular Probes/Invitrogen) such as QSY7, QSY9, QSY21, and QSY35, and other quenchers such as Dabcyl and Dabsyl, Cy5Q and Cy7Q and Dark Cyanine dyes (GE Healthcare), Dy-Quenchers (Dyomics), such as DYQ-660 and DYQ-661, ATTO fluorescent quenchers (ATTO-TEC GmbH), such as ATTO 540Q, 580Q, and 612Q, green fluorescent protein (GFP), yellow fluorescent protein (YFP), blue fluorescent protein (BFP), cyan fluorescent protein (CYP), and red fluorescent protein (RFP).

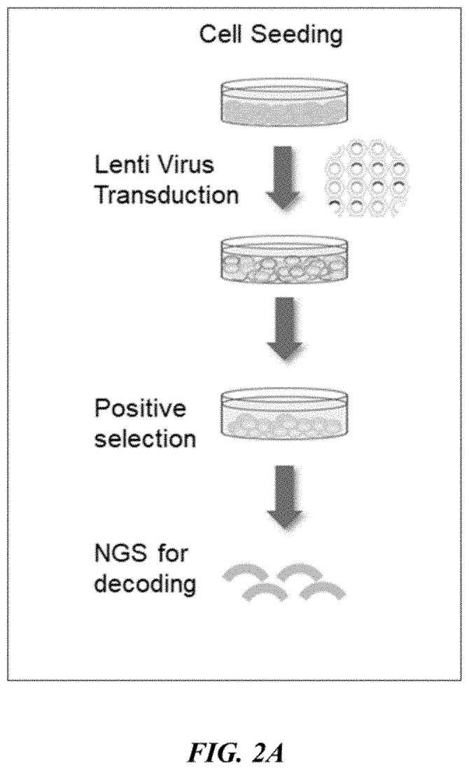

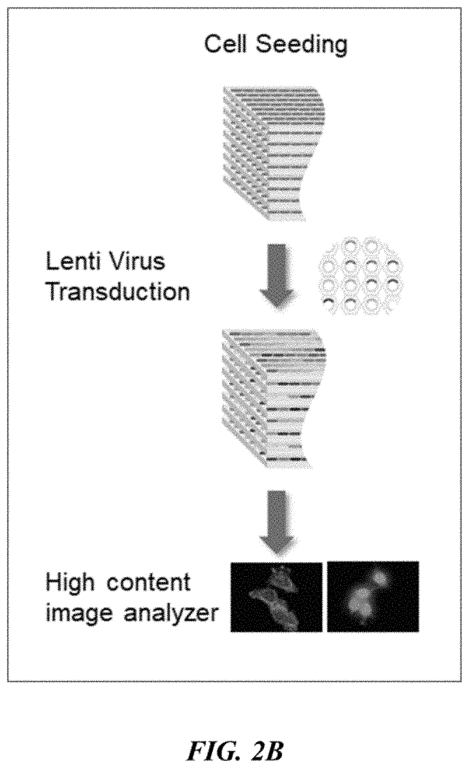

FIG. 2A shows an example of a method for pooled genetic screening. In pooled genetic screening, cell seeding and transduction (such as through use of a lentivirus) may produce pools of genetically-modified cells, each cell of which may comprise an exogenous nucleic acid molecule (such as an exogenous DNA molecule or an exogenous RNA molecule) from a library. The pool of genetically-modified cells may be heterogenous. A selection procedure may then be carried out and nucleic acids from cells of interest are sequenced by next-generation nucleic acid sequence methods. Pooled genetic screening may be a very powerful tool in some cases, such as in identifying genes responsible for pathways of interest. To date, selection methods have mostly relied on positive screening, negative screening, or fluorescence intensity-based screening by fluorescence-activated cell sorting (FACS).

FIG. 2B shows an example method of image-based genetic screening. In image-based screening, cell seeding and transduction produce genetically modified cells. In contrast to pooled genetic screening, however, in image-based screening the resultant genetically modified-cells are analyzed using imaging techniques, such as fluorescence imaging. Genetically modified cells may be screened using image-based screening to identify cells having a property of interest. Such image-based screening may provide detailed structural information about cells of interest, but may be time-consuming or costly.

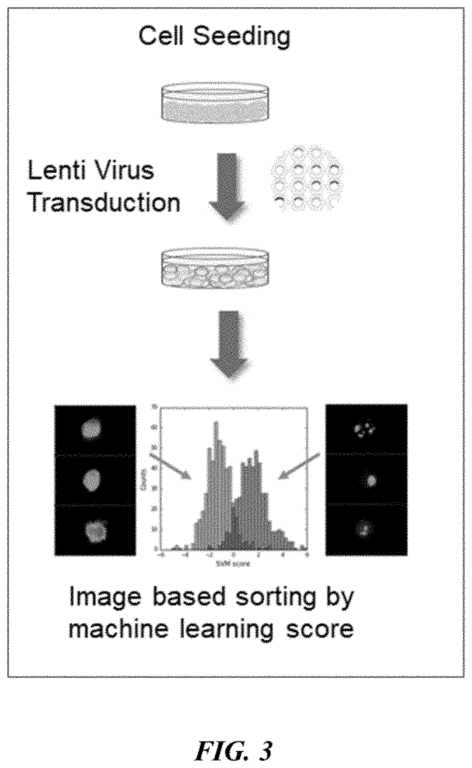

FIG. 3 shows an example method of image-based pooled genetic screening. The image-based pooled genetic screening procedure may utilize cell seeding and transduction (such as through use of a lentivirus) to produce pools of genetically modified cells, each cell of which may comprise an exogenous nucleic acid molecule (such as an exogenous DNA molecule or an exogenous RNA molecule) from a library. The pool of genetically-modified cells may be heterogenous. An imaging procedure (such as one or more imaging procedures disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, or one or more imaging procedures disclosed in N. Nitta, T. Sugimura, A. Isozaki, H. Mikami, S. Sakuma, T. Iino, F. Arai, T. Endo, Y. Fujiwaki, H. Fukuzawa, M. Hase, T. Hayakawa, K. Hiramatsu, Y. Hoshino, M. Inaba, T. Ito, H. Karakawa, Y. Kasai, K. Koizumi, S. Lee, C. Lei, M. Li, T. Maeno, S. Matsusaka, D. Murakami, A. Nakagawa, Y. Oguchi, M. Oikawa, T. Ota, K. Shiba, H. Shintaku, Y. Shirasaki, K. Suga, Y. Suzuki, N. Suzuki, Y. Tanaka, H. Tezuka, C. Toyokawa, Y. Yalikun, M. Yamada, M. Yamagishi, T. Yamano, A. Yasumoto, Y. Yatomi, M. Yazawa, D. Di Carlo, Y. Hosokawa, S. Uemura, Y. Ozeki, and K. Goda, “Intelligent Image-Activated Cell Sorting”, Cell 175(1), pp. 266-276 (2018), doi: 10.1016/j.cell.2018.08.028, which references are entirely incorporated herein by reference) may then carried out to select cells having a property of interest based on the information contained within the image (such as protein localization or protein aggregation within cells of interest). The cells having the property of interest may be sorted and nucleic acids of interest may be extracted from the cells having the property of interest. The nucleic acids of interest may then be sequenced by next-generation nucleic acid sequence methods. Image-based pooled screening may provide many advantages over the pooled screening and image-based screening methods described above.

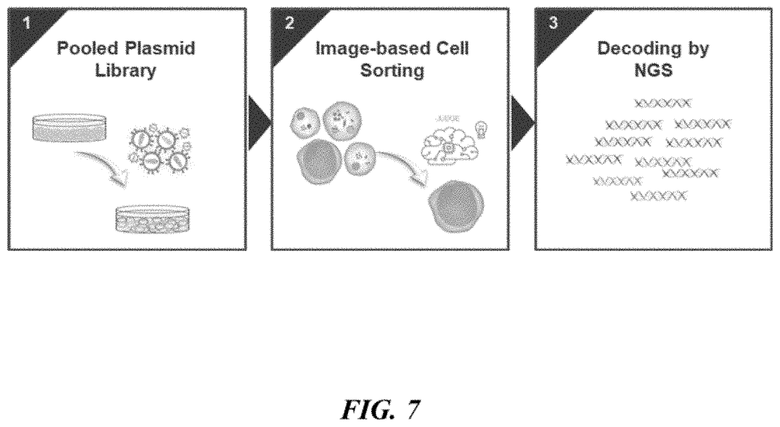

FIG. 7 shows an example method of target discovery using rapid image-based pooled genetic screening (RIPGS). In some cases, a morphological change may be identified in a cell that comprises an exogenous RNA molecule or an exogenous DNA molecule. In some cases, the morphological change may be identified as being caused by the exogenous RNA molecule or the exogenous DNA molecule. The identification of the morphological change may provide information about the exogenous RNA molecule or the exogenous DNA molecule, which may be identified as a target molecule. In some cases, a peptide or protein encoded by the exogenous RNA molecule or the exogenous DNA molecule may be a peptide or protein having therapeutic properties or may become a therapeutic drug. In some cases, a target may be a peptide or nucleic acid capable of effecting a morphological change of a cell. According the present invention, the morphological change may comprise one or more members selected from the group consisting of: a change in a protein-protein interaction within the cell, a change in protein localization within the cell, a change in shape of the cell, a change in shape of one or more components of the cell, and a change in shape of one or more organelles of the cell. In a first operation, illustrated in panel 1, the method may comprise transfecting a pooled plasmid library into cells to produce a pool of cells comprising genetically-modified cells. For example, the pooled plasmid library may comprise plasmids encoding one or more members selected from the group consisting of: a peptide, a protein, a polypeptide, a non-coding RNA, a gRNA, an sgRNA, an shRNA, an siRNA, an miRNA, or an asRNA. The pool of cells may be heterogeneous. In some cases, plasmids in the pooled plasmid library may encode potential target candidates.

In a second operation, illustrated in panel 2 of FIG. 7, the method may comprise isolating cells with target phenotypes (for instance, based on whether an image of the cells shows protein localization, protein-protein interactions, translocation, co-localization, puncta, or other attributes). Isolating the cells may comprise sorting the cells. In some cases, isolating the cells may comprise image-based cell sorting. The target phenotype may comprise a morphological change of the cell. The cells may be isolated based on the morphological change of the cell. The target phenotype may be effected by a plasmid, or a product encoded by the plasmid, from the pooled plasmid library.

In a third operation, illustrated in panel 3 of FIG. 7, the method may comprise identifying the inserted plasmid by nucleic acid sequencing. The nucleic acid sequencing may comprise DNA sequencing or RNA sequencing. The nucleic acid sequencing may comprise reverse transcription followed by DNA sequencing. The nucleic acid sequencing may comprise massively parallel array sequencing. The nucleic acid sequencing may comprise sequencing by synthesis. The nucleic acid sequencing may comprise next-generation sequencing. The method may be applied to numerous applications, including target identification, or screening functional hit molecules inside cells. In general, the method may be regarded as a method for high-throughput, high-content screening.

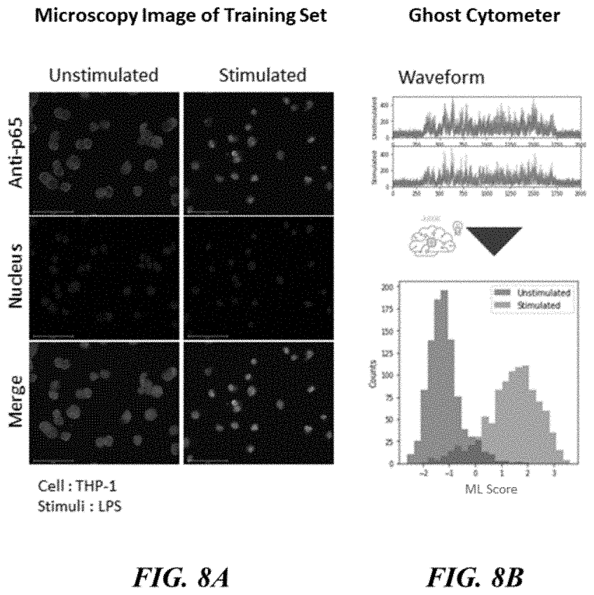

Isolation of cells having a target phenotype, for example, as performed at the second operations of and FIG. 7, may comprise sorting cells using rapid image-based pooled genetic/peptide screening (RIPGS). RIPGS may comprise training a machine learning algorithm to differentiate cells based on the presence or absence of the target phenotype. The machine learning algorithm may be trained using a training set, as illustrated in FIG. 8A and FIG. 8B. In some cases, a training set may comprise a positive control data set and a negative control data set. The positive control data set may comprise cells having the target phenotype. The negative control data set may comprise cells lacking the target phenotype. For example, the positive control data set may comprise cells treated with a reagent to induce the target phenotype. The positive training set and the negative training set may be imaged, for example using fluorescence microscopy, as shown in FIG. 8A. Waveforms for individual cells from each of the positive control data set and the negative control data set may be measured, as shown in the top panel of FIG. 8B. The waveforms may be used to train a machine learning algorithm and assign machine learning (ML) scores to measured cell waveforms, as shown in the bottom panel of FIG. 8B. A ML score determined from a cell waveform may indicate a similarity of a cell to the target phenotype. The trained machine learning algorithm may be used to assign a ML score to individual cells from a pool of cells, for example a pool of cells comprising genetically-modified cells. Individual cells form the pool of cells may be sorted based on assigned ML scores. In some cases, isolation of cells may be performed using ghost cytometry, as described in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference.

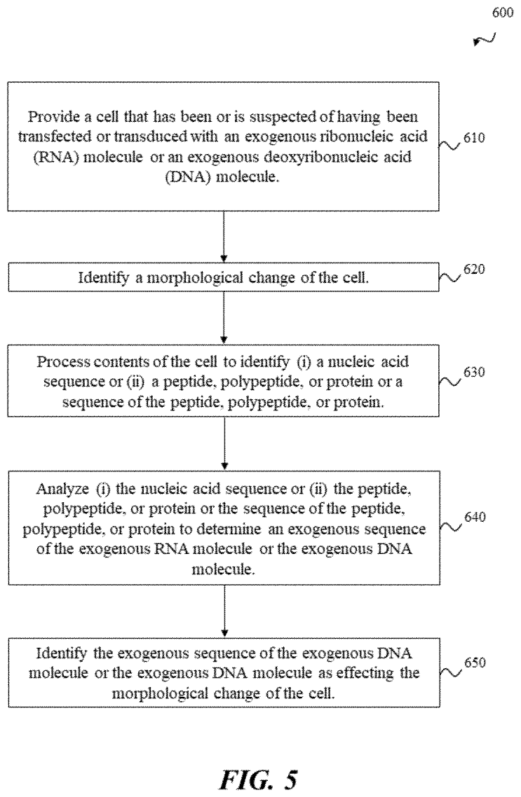

FIG. 5 shows a flowchart for an example of a method 600 for identifying a nucleic acid molecule.

In a first operation 610, the method 600 may comprise providing a cell that has been or is suspected of having been transfected or transduced with at least one exogenous RNA or at least one exogenous DNA molecule. An exogenous sequence of the exogenous RNA molecule or the exogenous DNA molecule may be unknown. An exogenous sequence of the exogenous RNA molecule or the exogenous DNA molecule may be known. The exogenous RNA molecule or the exogenous DNA molecule may encode a gene, peptide, polypeptide, or protein.

The cell may be derived from any source. For instance, the cell may be a human cell, an animal cell, a non-human primate cell, an equine cell, a porcine cell, a canine cell, a feline cell, a murine cell, a plant cell, a bacterial cell, or any other cell. The cell may derive from a tissue or from an organ. For instance, the cell may be derived from a heart, artery, vein, brain, nerve, lung, spine, spinal cord, bone, connective tissue, trachea, esophagus, stomach, small or large intestine, bladder, liver, kidney, spleen, urethra, ureter, prostate, vas deferens, penis, ovary, uterus, endometrium, fallopian tube, or vagina, or any tissue associated with any of the preceding. The cell may be or may be suspected of being a cancerous cell (such as a tumor cell). For example, the cell may derive from tumor tissue. The cell may include natural or non-natural components. For example, the cell may include nucleic acids (such as DNA or RNA), peptides, polypeptides, proteins, lipids, or carbohydrates. The cell may include one or more optically detectable elements such as one or more fluorophores. The fluorophores may be native or non-native to the cell. For instance, the fluorophores may be non-native fluorophores that have been introduced to the cell, such as by one or more cell staining or labeling techniques.

The cell may be among a population of cells. For instance, the cell may be among a population of at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, 100,000,000, 200,000,000, 300,000,000, 400,000,000, 500,000,000, 600,000,000, 700,000,000, 800,000,000, 900,000,000, 1,000,000, or more cells. The cell may be among a population of at most about 1,000,000,000, 900,000,000, 800,000,000, 700,000,000, 600,000,000, 500,000,000, 400,000,000, 300,000,000, 200,000,000, 100,000,000, 90,000,000, 80,000,000, 70,000,000, 60,000,000, 50,000,000, 40,000,000, 30,000,000, 20,000,000, 10,000,000, 9,000,000, 8,000,000, 7,000,000, 6,000,000, 5,000,000, 4,000,000, 3,000,000, 2,000,000, 1,000,000, 900,000, 800,000, 700,000, 600,000, 500,000, 400,000, 300,000, 200,000, 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, 1, or fewer cells. The cell may be among a population of cells that is within a range defined by any two of the preceding values. The cell may be among a population of from 1 to 1,000,000 cells, from 10 to 1,000,000 cells, from 100 to 1,000,000 cells, from 1,000 to 1,000,000 cells, from 10,000 to 1,000,000 cells, from 100,000 to 1,000,000 cells, from 1 to 100,000 cells, from 10 to 100,000 cells, from 100 to 100,000 cells, from 1,000 to 100,000 cells, from 10,000 to 100,000 cells, from 1 to 10,000 cells, from 10 to 10,000 cells, from 100 to 10,000 cells, from 1,000 to 10,000 cells, from 1 to 1,000 cells, from 10 to 1,000 cells, from 100 to 1,000 cells, from 1 to 100 cells, from 10 to 100 cells, or from 1 to 10 cells. The cell may be isolated from the population of cells. The cell may be isolated from a plurality of cells.

The cell or population of cells may be immobilized or fixed. The cell or population of cells may be not immobilized or not fixed. The cell or population of cells may be at rest. The cell or population of cells may be in motion. For instance, the cell or population of cells may be flowing (such as through or in a flow channel or flow cell).

The at least one exogenous RNA molecule or at least one exogenous DNA molecule may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1,000, or more exogenous RNA molecules or exogenous DNA molecules. The at least one exogenous RNA molecule or at least one exogenous DNA molecule may comprise at most about 1,000, 900, 800, 700, 600, 500, 400, 300, 200, 100, 90, 80, 70, 60, 50, 40, 30, 20, 10, 9, 8, 7, 6, 5, 4, 3, 2, 1, or fewer exogenous RNA molecules or exogenous DNA molecules. The at least one exogenous RNA molecule or at least one exogenous DNA molecule may comprise a number of exogenous RNA molecules or exogenous DNA molecules that is within a range defined by any two of the preceding values. The at least one exogenous RNA molecule or at least one exogenous DNA molecule may comprise from 1 to 1,000, from 10 to 1,000, from 100 to 1,000, from 1 to 100, from 10 to 100, from 1 to 10, from 1 to 5, from 5 to 10, from 10 to 20, or from 1 to 20 exogenous RNA molecules or exogenous DNA molecules.

The cell may be transfected using a plasmid. The plasmid may comprise the exogenous RNA molecule or the exogenous DNA molecule.

In a second operation 620, the method 600 may comprise identifying a morphological change of the cell. The morphological change may comprise one or more members selected from the group consisting of: a change in a protein-protein interaction within the cell, a change in protein localization within the cell, a change in shape of the cell, a change in shape of one or more components of the cell (such as a change in shape of cytoplasm within the cell or a change in shape or arrangement of the cytoskeleton), and a change in shape of one or more organelles of the cell (such as a change in shape of a nucleolus, a nucleus, a ribosome, a vesicle, a rough endoplasmic reticulum, a Golgi apparatus, a cytoskeleton, a smooth endoplasmic reticulum, a mitochondrion, a vacuole, a lysosome, a centrosome, or a cell membrane of the cell).

The morphological change may be identified using one or more optical techniques, such as optical imaging, optical microscopy, or optical spectroscopy. The morphological change may be identified using one or more of bright-field microscopy, cross-polarized microscopy, dark-field microscopy, phase contrast microscopy, differential interference contrast (DIC) microscopy, reflected interference microscopy, Sarfus microscopy, fluorescence microscopy, epifluorescence microscopy, confocal microscopy, light sheet microscopy, multi-photon microscopy, super-resolution microscopy, near-field scanning optical microscopy, near-field optical random mapping (NORM) microscopy, structured illumination microscopy (SIM), spatially-modulated illumination (SMI) microscopy, 4-pi microscopy, spectral precision distance microscopy, stimulated emission depletion (STED) microscopy, ground-state depletion (GSD) microscopy, reversible saturable optical linear fluorescence transition (RESOLFT) microscopy, binding-activated localization (BALM) microscopy, photo-activated localization (PALM) microscopy, stochastic optical reconstruction (STORM) microscopy, direct stochastic optical reconstruction microscopy (dSTORM), super-resolution optical fluctuation (SOFI) microscopy, or omnipresent localization microscopy (OLM).

The morphological change may be identified by obtaining temporal signals containing image information. The temporal signals may be transformed into time-independent image information. The morphological change may be identified without reconstructing an image. For example, the morphological change may be identified without obtaining a full image of the cell. Identifying the morphological change may comprise obtaining temporal signals containing structural information. The temporal signals may be transformed into time-independent structural information. Identifying the morphological change may comprise processing a temporal waveform signal obtained by effectively compressing cell spatial information in the optical imaging method instead of two-dimensional information of the cell image. The morphological change may be identified using a trained machine learning algorithm. The trained machine learning algorithm may be trained using teacher data. For example, the teacher data may comprise image, structural, or temporal data of cells having a known morphological property. For instance, the morphological change may be identified using one or more of the imaging methods and systems disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference.

The morphological change may be identified while the cell is immobilized or fixed. The morphological change may be identified while the cell is not immobilized or not fixed. The morphological change may be identified while the cell is at rest. The morphological change may be identified while the cell is in motion. For instance, the morphological change may be identified while the cell is flowing (such as through or in a flow channel or flow cell).

A machine learning algorithm may be used to identify the morphological change. For instance, the machine learning algorithm may be trained using a training set comprising labeled or unlabeled images of cell populations that feature a cell that displays one or more features of interest (such as one or more particular protein localization patterns, protein-protein interactions, translocations, co-localization, puncta, or other attributes, as described herein). The machine learning algorithm may then be applied to test images to classify a cell within a population of cells as positive or negative for the one or more features of interest.

The machine learning algorithm may comprise one or more supervised, semi-supervised, or unsupervised machine learning techniques. The machine learning algorithm may comprise one or more of regression analysis, regularization, classification, dimensionality reduction, ensemble learning, meta learning, reinforcement learning, association rule learning, cluster analysis, anomaly detection, deep learning, or ultra-deep learning. The machine learning algorithm may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principle component regression, least absolute shrinkage and selection operation, least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

The machine learning algorithm may classify the cell as positive or negative for the one or more features with one or more of a sensitivity, specificity, and an accuracy of at least about 80%, 81%, 82%, 83%, 84%, 85%, 86%, 87%, 88%, 89%, 90%, 91%, 92%, 93%, 94%, 95%, 96%, 97%, 98%, 99%, or more. The machine learning algorithm may classify the cell as positive or negative for the one or more features with one or more of a sensitivity, specific, and an accuracy that is within a range define by any two of the preceding values.

The cell may be isolated upon or subsequent to identifying the morphological change. For instance, the cell may be isolated using flow cytometry or one or more of the “ghost cytometry” methods and systems disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference.

Prior to second operation 620, the method may further comprise labeling peptides, polypeptides, and/or proteins within the cell. The labeling may comprise labeling the peptides, polypeptides, and/or proteins within the cell with one or more fluorescent labels, Forster resonance energy transfer (FRET) labels, dyes, fluorophores, quantum dots, or any other labels. For instance, the labeling may comprise labeling the peptides, polypeptides, and/or proteins within the cell with one or more of SYBR green, SYBR blue, 4′,6-diamidino-2-phenylindole (DAPI), propidium iodine, Hoechst, SYBR gold, ethidium bromide, acridine, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and 9-amino-6-chlor-2-methoxyacridine (ACMA), Hoechst 33258, Hoechst 33342, Hoechst 34580, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, and -45 (blue), SYTO-13, -16, -24, -21, -23, -12, -11, -20, -22, -15, -14, and -25 (green), SYTO-81, -80, -82, -83, -84, and -85 (orange), SYTO-64, -17, -59, -61, -62, -60, and -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), SYBR Green I, SYBR Green II, SYBR Gold, CellTracker Green, 7-aminoactinomycin D (7-AAD), ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-amino-4-methylcoumarin-3-acetic acid (AMCA), BODIPY fluorophores, 8-methoxypyrene-1,3,6-trisulfonic acid trisodium salt, 3,6-disulfonate-4-amino-naphthalimide, phycobiliproteins, Atto 390, 425, 465, 488, 495, 532, 565, 594, 633, 647, 647N, 665, 680, and 700 dyes, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BH1-0, BHQ-1, BHQ-3, and BHQ-10, QSY Dye fluorescent quenchers (from Molecular Probes/Invitrogen) such as QSY7, QSY9, QSY21, and QSY35, and other quenchers such as Dabcyl and Dabsyl, Cy5Q and Cy7Q and Dark Cyanine dyes (GE Healthcare), Dy-Quenchers (Dyomics), such as DYQ-660 and DYQ-661, ATTO fluorescent quenchers (ATTO-TEC GmbH), such as ATTO 540Q, 580Q, and 612Q, green fluorescent protein (GFP), yellow fluorescent protein (YFP), blue fluorescent protein (BFP), cyan fluorescent protein (CYP), red fluorescent protein (RFP), and others. In some cases, the label may comprise one or more linkers. For instance, a label may have a disulfide linker attached to the label. Non-limiting examples of such labels include Cy5-azide, Cy-2-azide, Cy-3-azide, Cy-3.5-azide, Cy5.5-azide and Cy-7-azide. In some cases, a linker may comprise a cleavable linker. In some cases, the label may be configured such that the label does not self-quench or exhibit proximity quenching. Non-limiting examples of a label type that does not self-quench or exhibit proximity quenching include bimane derivatives such as Monobromobimane. Alternatively, the label may be configured such that the label self-quenches or exhibits proximity quenching. Non-limiting examples of such labels include Cy5-azide, Cy-2-azide, Cy-3-azide, Cy-3.5-azide, Cy5.5-azide and Cy-7-azide.

In a third operation 630, the method 600 may comprise processing contents of the cell to identify a nucleic acid sequence or a peptide, polypeptide, or protein, or a sequence of the peptide, polypeptide, or protein. The operation 630 may comprise sequencing at least one nucleic acid molecule and/or at least one peptide, polypeptide, or protein molecule from the cell. The method may comprise sequencing at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, 100,000,000, 200,000,000, 300,000,000, 400,000,000, 500,000,000, 600,000,000, 700,000,000, 800,000,000, 900,000,000, 1,000,000,000, or more nucleic acid molecules or peptide, polypeptide, or protein molecules from the cell. The method may comprise sequencing at most about 1,000,000,000, 900,000,000, 800,000,000, 700,000,000, 600,000,000, 500,000,000, 400,000,000, 300,000,000, 200,000,000, 100,000,000, 90,000,000, 80,000,000, 70,000,000, 60,000,000, 50,000,000, 40,000,000, 30,000,000, 20,000,000, 10,000,000, 9,000,000, 8,000,000, 7,000,000, 6,000,000, 5,000,000, 4,000,000, 3,000,000, 2,000,000, 1,000,000, 900,000, 800,000, 700,000, 600,000, 500,000, 400,000, 300,000, 200,000, 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or 1 nucleic acid molecules or peptide, polypeptide, or protein molecules from the cell. The method may comprise sequencing a number of nucleic acid molecules or peptide, polypeptide, or protein molecules from the cell that is within a range defined by any two of the preceding values. The method may comprise sequencing at from 1 to 1,000,000,000, from 10 to 1,000,000,000, from 100 to 1,000,000,000, from 1,000 to 1,000,000,000, from 10,000 to 1,000,000,000, from 100,000 to 1,000,000,000, from 1,000,000 to 1,000,000,000, from 10,000,000 to 1,000,000,000, from 100,000,000 to 1,000,000,000, from 1 to 100,000,000, from 10 to 100,000,000, from 100 to 100,000,000, from 1,000 to 100,000,000, from 10,000 to 100,000,000, from 100,000 to 100,000,000, from 1,000,000 to 100,000,000, from 10,000,000 to 100,000,000, from 1 to 10,000,000, from 10 to 10,000,000, from 100 to 10,000,000, from 1,000 to 10,000,000, from 10,000 to 10,000,000, from 100,000 to 10,000,000, from 1,000,000 to 10,000,000, from 1 to 1,000,000, from 10 to 1,000,000, from 100 to 1,000,000, from 1,000 to 1,000,000, from 10,000 to 1,000,000, from 100,000 to 1,000,000, from 1 to 100,000, from 10 to 100,000, from 100 to 100,000, from 1,000 to 100,000, from 10,000 to 100,000, from 1 to 10,000, from 10 to 10,000, from 100 to 10,000, from 1,000 to 10,000, from 1 to 1,000, from 10 to 1,000, from 100 to 1,000, from 1 to 100, from 10 to 100, or from 1 to 10 nucleic acid molecules or peptide, polypeptide, or protein molecules from the cell.

The sequencing may comprise any nucleic acid sequencing, such as any DNA sequencing or RNA sequencing. The sequencing may comprise polymerase chain reaction (PCR), digital PCR, real-time PCR, quantitative PCR (qPCR), reverse transcription, reverse-transcription PCR (RT-PCR), Sanger sequencing, high-throughput sequencing, sequencing-by-synthesis, single-molecule sequencing, sequencing-by-ligation, RNA-Seq (Illumina), next-generation sequencing, Digital Gene Expression (Helicos), array hybridization, Clonal Single MicroArray (Solexa), shotgun sequencing, Maxim-Gilbert sequencing, massively parallel sequencing, or massively parallel array sequencing.

In a fourth operation 640, the method 600 may comprise analyzing the nucleic acid sequence or the peptide, polypeptide, or protein or the sequence of the peptide, polypeptide, or protein to determine the exogenous sequence of the exogenous RNA or exogenous DNA.

In a fifth operation 650, the method 600 may comprise identifying the exogenous sequence of the exogenous RNA or exogenous DNA molecule as effecting the morphological change of the cell.

The method 600 may further comprise using at least the morphological change to determine that the at least one exogenous DNA molecule or at least one exogenous RNA molecule inhibits or activates a biochemical pathway within the cell.

In an aspect, the present disclosure provides a method for screening a cell for a target nucleic acid, peptide, polypeptide, or protein comprising: (a) providing a cell that has been transfected or transduced with an exogenous ribonucleic acid (RNA) molecule or an exogenous deoxyribonucleic (DNA) molecule, wherein the exogenous RNA or DNA encoded to express a population of macromolecules within the cell, (b) identifying a morphological change of the cell in response to the population of macromolecules expressed by the cell, (c) subjecting a genome of the cell to sequence identification to identify the exogenous RNA or DNA, and (d) determining that the exogenous RNA or DNA induced the morphological change. Identifying the morphological change may comprise identifying a localization profile or pattern of macromolecules of the population of macromolecules. Identifying the morphological change may comprise identifying a shape of the cell or one or more sub-cellular constituents of the cell. The one or more sub-cellular constituents may comprise an organelle of the cell. The population of macromolecules may comprise a population of one or more members selected from the group consisting of peptides, polypeptides, proteins, and nucleic acid molecules.

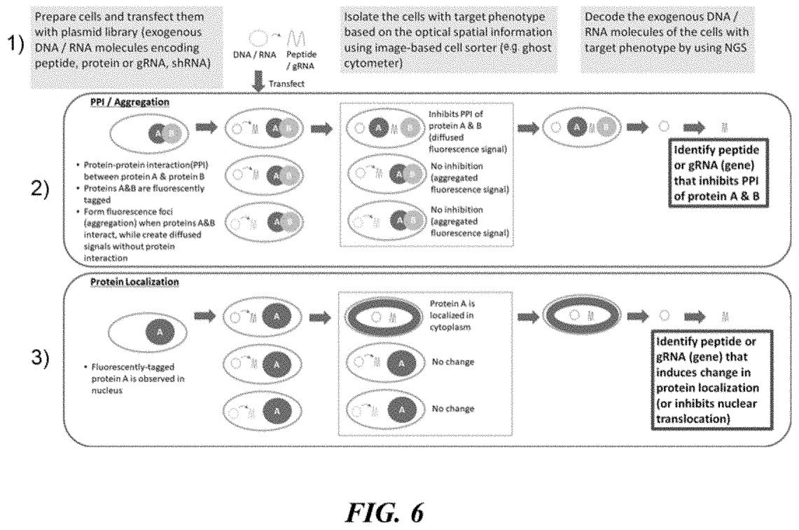

FIG. 6 shows an example of a method for identifying a nucleic acid molecule or a peptide, polypeptide, or protein based on protein-protein interactions, protein aggregation, or protein localization. As shown in panel 1 of FIG. 6, cells may be prepared and transfected with a plasmid library comprising exogenous DNA or RNA molecules, as described herein. The exogenous DNA or RNA molecules may encode for peptides, polypeptides, proteins, or nucleic acids such as RNA, DNA, short hairpin RNA (shRNA), guide RNA (gRNA), small interfering RNA (siRNA), micro RNA (miRNA), antisense RNA (asRNA) or guide DNA (gDNA), as described herein. Cells displaying a target phenotype may be isolated based on optical spatial information, as described herein. The exogenous DNA or RNA molecules associated with the cells displaying the target phenotype may then be decoded using nucleic acid sequencing, such as next-generation sequencing (NGS), as described herein.

As shown in panel 2 of FIG. 6, protein-protein interactions (PPI) between a first protein A and a second protein B may give rise to morphological changes that may be detected. The proteins A and B may be fluorescently tagged. The proteins A and B may form fluorescent foci when the proteins A and B interact. When proteins A and B do not interact, they may not form such fluorescent foci. The presence or absence of such fluorescent foci may be detected using the optical detection systems and methods described herein. One or more of the exogenous DNA or RNA molecules (or a peptide, polypeptide, or protein expressed from the exogenous DNA or RNA molecules) described herein may inhibit formation of the fluorescent foci, while other exogenous DNA or RNA molecules (or a peptide, polypeptide, or expressed from the exogenous DNA or RNA molecules) may not inhibit formation of the fluorescent foci. Thus, cells transfected with exogenous DNA or RNA molecules that inhibit formation of the fluorescent foci (e.g., exogenous DNA or RNA molecules that inhibit PPI, or a peptide, polypeptide, or protein expressed from the exogenous DNA or RNA molecules that inhibits PPI) may be distinguished from cells transfected with exogenous DNA or RNA molecules that do not inhibit formation of the fluorescent foci (e.g., exogenous DNA or RNA molecules that do not inhibit PPI or a peptide, polypeptide, or protein expressed from the exogenous DNA or RNA molecules that does not inhibit PPI). The exogenous DNA or RNA molecules (or a peptide, polypeptide, or protein expressed from the exogenous DNA or RNA molecules) that inhibit PPI may then be isolated and subjected to nucleic acid sequencing to identify the peptide, polypeptide, protein, or nucleic acid that inhibits PPI between proteins A and B.

In some cases, one or more of the exogenous DNA or RNA molecules (or a peptide, polypeptide, or protein expressed from the exogenous DNA or RNA molecules) described herein may promote formation of the fluorescent foci, while other exogenous DNA or RNA molecules (or a peptide, polypeptide, or expressed from the exogenous DNA or RNA molecules) may not promote formation of the fluorescent foci. Thus, cells transfected with exogenous DNA or RNA molecules that promote formation of the fluorescent foci (e.g., exogenous DNA or RNA molecules that promote PPI, or a peptide, polypeptide, or protein expressed from the exogenous DNA or RNA molecules that promotes PPI) may be distinguished from cells transfected with exogenous DNA or RNA molecules that do not promote formation of the fluorescent foci (e.g., exogenous DNA or RNA molecules that do not promote PPI or a peptide, polypeptide, or protein expressed from the exogenous DNA or RNA molecules that does not promote PPI). The exogenous DNA or RNA molecules (or a peptide, polypeptide, or protein expressed from the exogenous DNA or RNA molecules) that promote PPI may then be isolated and subjected to nucleic acid sequencing to identify the peptide, polypeptide, protein, or nucleic acid that promotes PPI between proteins A and B.

As shown in panel 3 of FIG. 6, protein localization may give rise to morphological changes that may be detected. Protein A may be fluorescently tagged. In some cases, protein A may be detected in the nucleus of a cell.

In some cases, one or more of the exogenous DNA or RNA molecules (or a peptide, polypeptide, or protein expressed by a cell in response to the exogenous DNA or RNA molecules) described herein may cause protein A to become localized in the cytoplasm of a cell, while other exogenous DNA or RNA molecules (or a peptide, polypeptide, or expressed from the exogenous DNA or RNA molecules) may not cause such localization. Thus, cells transfected with exogenous DNA or RNA molecules that cause such protein localization (e.g., exogenous DNA or RNA molecules that cause protein localization, or a peptide, polypeptide, or protein expressed from the exogenous DNA or RNA molecules that cause protein localization) may be distinguished from cells transfected with exogenous DNA or RNA molecules that do not cause such protein localization (e.g., exogenous DNA or RNA molecules that do not cause protein localization or a peptide, polypeptide, or protein expressed from the exogenous DNA or RNA molecules that does not cause protein localization). The exogenous DNA or RNA molecules (or a peptide, polypeptide, or protein expressed from the exogenous DNA or RNA molecules) that cause protein localization may then be isolated and subjected to nucleic acid sequencing to identify the peptide, polypeptide, protein, or nucleic acid that cause protein localization.

In some cases, one or more of the exogenous DNA or RNA molecules (or a peptide, polypeptide, or protein expressed by a cell in response to the exogenous DNA or RNA molecules) described herein may cause a change in localization of protein A. For example, one or more of the exogenous DNA or RNA molecules (or a peptide, polypeptide, or protein expressed by a cell in response to the exogenous DNA or RNA molecules) described herein may cause the localization of protein A to change from a first organelle or subcellular region or compartment to a second organelle or subcellular region or compartment. The first organelle or subcellular region or compartment may comprise a mitochondrion, a nucleus, a cytoplasm, a cytoskeleton, a cell membrane, a nuclear membrane, a lysosome, an endosome, an endoplasmic reticulum, a Golgi apparatus, a vacuole, a lysosome, a centrosome, a nucleolus, a ribosome, or a vesicle. The second organelle or subcellular region or compartment may comprise a mitochondrion, a nucleus, a cytoplasm, a cytoskeleton, a cell membrane, a nuclear membrane, a lysosome, an endosome, an endoplasmic reticulum, a Golgi apparatus, a vacuole, a lysosome, a centrosome, a nucleolus, a ribosome, or a vesicle.

Computer systems

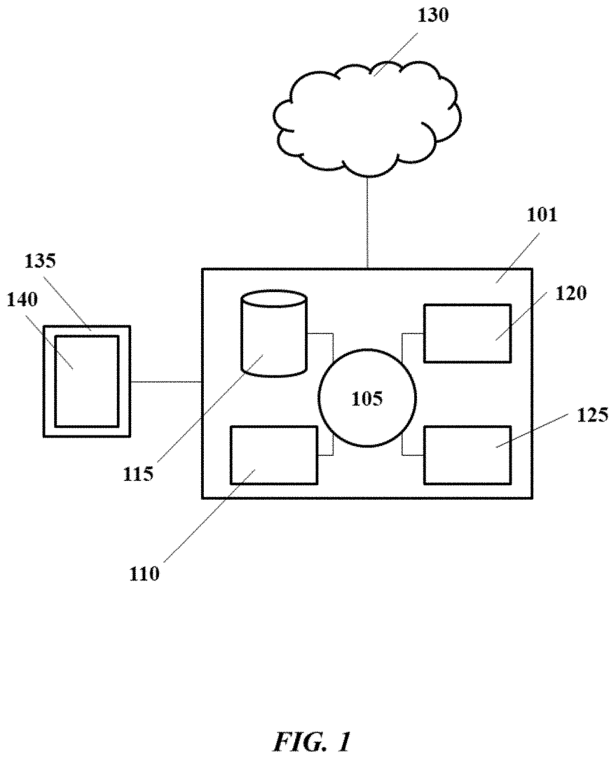

The present disclosure provides computer systems that are programmed to implement methods of the disclosure. FIG. 1 shows a computer system 101 that is programmed or otherwise configured to screen a biological sample for one or more targets, such as a peptide, polypeptide, protein, or organelle, or one or more target phenotypes, such as a protein localization, a protein-protein interaction, or a cell morphology.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The electronic storage unit 115 can store files, such as drivers, libraries and saved programs. The electronic storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, an image(s) of a cell(s), including an peptide, polypeptide and/or protein (e.g., protein distribution), and/or sequencing information from a genome (deoxyribonucleic acid and/or ribonucleic acid) from a cell. Examples of UP s include, without limitation, a graphical user interface (GUI) and web-based user interface.

Methods and systems of the present disclosure can be implemented by way of one or more algorithms. An algorithm can be implemented by way of software upon execution by the central processing unit 105.

### EXAMPLES

### Example 1: Experimental Protocols

In some cases, the exogenous RNA molecule or exogenous DNA molecule is designed to express peptides, polypeptides, or proteins. The peptides, polypeptides, or proteins may have a stable a-helical structure, which may be a critical aspect of peptide-protein interactions. The peptide expression RNA or DNA may be cloned to plasmid vectors for transfection or transduction into a cell.

Two peptide expression libraries were constructed with tailored randomization of 30 residues. The scaffold peptide was designed to have a stable secondary structure, having 8 helical turns. An extensive network of salt bridges on their surface between lysine (K) and glutamic acid (E) residues may stabilize the helical structure and may also help to increase the peptide solubility as reported. Alanine residues were placed to promote the formation of (internal) helical structures. The libraries were constructed by randomizing seven positions (for each library) of the alanine residues which located within the same planar surface of the helical structure, as detailed in Krstenansky J. L., Owen T J., Hagaman K A., McLean L R., FEBS Lett., 1989, 242(2):409-13. doi.org/10.1016/0014-5793(89)80512-5. Short model peptides having a high a-helical tendency: Design and solution properties, which is entirely incorporated herein by reference.

In some cases, the exogenous RNA molecule or exogenous DNA molecule is transfected into the cell using clustered regularly interspaced short palindromic repeats (CRISPR), such as CRISPR-associated 9 (Cas9), Cas13d, CasX or other variants, or other gene editing technologies, such as Zinc-finger nucleases (ZFN) or transcription activator-like effector nuclease (TALEN), as described in Shalem O., Sanjana NE., Hartenian E., Shi X., Scott D A., Mikkelson T., Heckl D., Ebert B L., Root D E., Doench J G., Zhang F., Science. 2014, 343 (6166):84-87. doi: 10.1126/science.1247005. Genome-scale CRISPR-Cas9 knockout screening in human cells; and Joung J., Konermann S., Gootenberg J S., Abudayyeh O O., Platt R J., Brigham M D., Sanjana N E., Zhang F., Nat Protoc. 2017, 12(4):828-863. doi: 10.1038/nprot.2017.016. Genome-scale CRISPR-Cas9 knockout and transcriptional activation screening, each of which is entirely incorporated herein by reference.

In some cases, a genome scale CRISPR library or peptide expression library is amplified. In brief, appropriate amount of plasmids were electroporated into electrocompetent cells. After confirming the transformation efficiency, amplified single guide RNA (sgRNA) library or peptide expression library were harvested and purified using the Macherey-Nagel NucleoBond Xtra Maxi EF Kit. Then, the sgRNA or sequences comprising peptide coding regions within the plasmids were PCR-amplified and barcoded before being subjected to high-throughput sequencing analysis before knockout experiments.

In some cases, the oligos of the peptide libraries were cloned into viral or non-viral vectors such as the pLVX-Puro lentiviral expression vectors. These vectors may provide a high-level expression of peptides in virtually any type mammalian cell type. Lentiviral particles for peptide libraries and sgRNA were manufactured with Lenti-X Packaging Single Shots kit (TaKara) using HEK 293FT cells. To ensure that most cells received only one genetic perturbation, the lentiviral particles (with sgRNA/peptide libraries) transduced to cell lines at an MOI (multiplicity of infection)<0.05 for the screening set.

Plasmid sequences of the cells were PCR amplified, purified, and subjected to de novo sequencing. The deep sequencing approach with next generation sequencing platform was used to decode the inserted sgRNA and plasmid distribution in the sorted cells.

### Example 2: Classification of Fluorescence-Tagged Mitochondria or Lysosomes

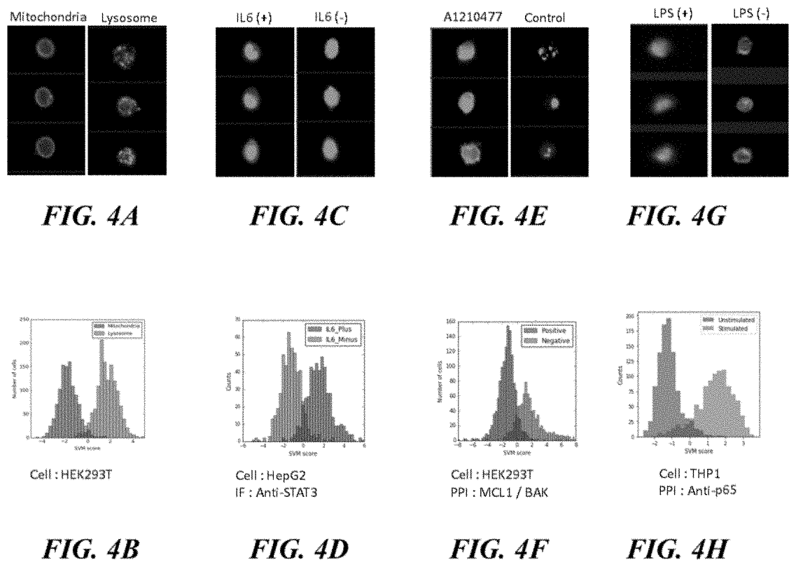

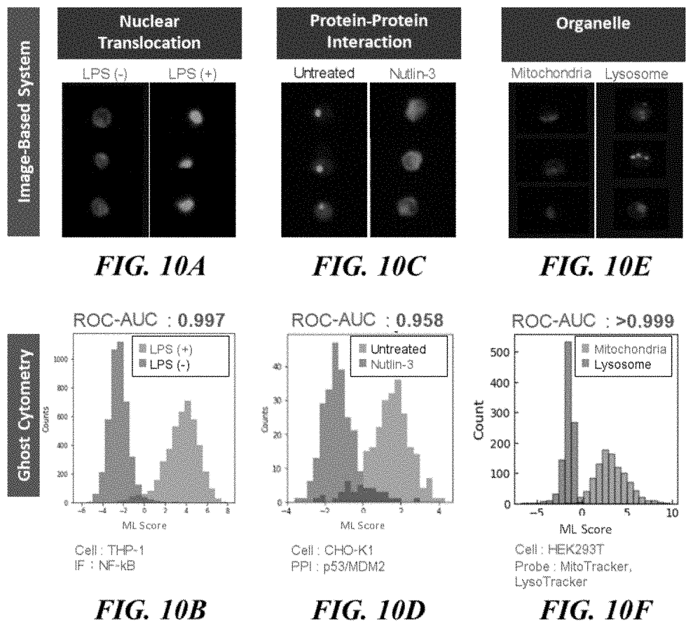

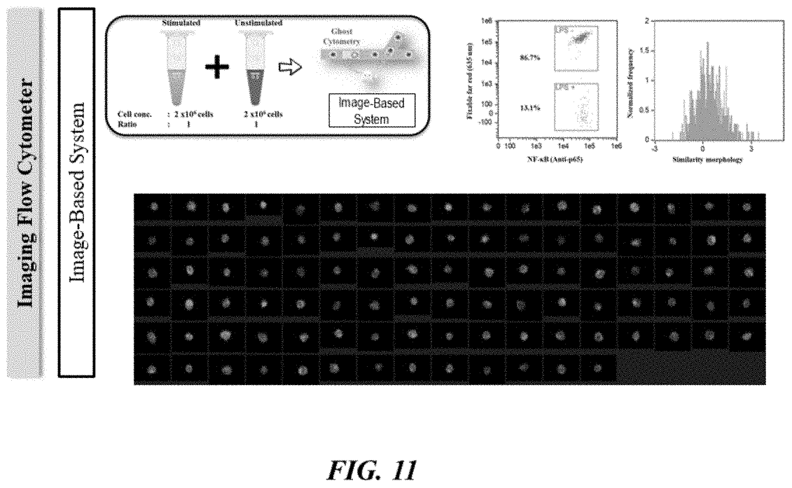

FIG. 4A and FIG. 10E show fluorescence images of HEK293T cells comprising fluorescently-tagged mitochondria or fluorescently-tagged lysosomes. The left panels in FIG. 4A and FIG. 10E show cells comprising mitochondria stained with Mito Tracker. The right panels in FIG. 4A and FIG. 10E show cells comprising lysosomes stained with Lyso Tracker. Cells stained with Mito Tracker or cells stained with Lyso tracker were used as a training set. Cells stained with Mito Tracker and cells stained with Lyso Tracker were differentiated by visual analysis using fluorescence imaging.

FIG. 4B shows classification of HEK293T cells comprising fluorescently-tagged mitochondria (left peak, “mitochondria”) or fluorescently-tagged lysosomes (right peak, “lysosomes”) using systems and methods of the present disclosure. FIG. 10F shows classification of HEK293T cells comprising fluorescently-tagged mitochondria (right peak, “mitochondria”) or fluorescently-tagged lysosomes (left peak, “lysosomes”) using systems and methods of the present disclosure. Cells stained with Mito Tracker and cells stained with Lyso Tracker were classified based on the morphology of the fluorescently-tagged organelles, thereby distinguishing between cells comprising fluorescently-tagged mitochondria and cells comprising fluorescently-tagged lysosomes. The proteins were classified using the imaging procedures disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference. The two peaks shown in each of FIG. 4B and FIG. 10F illustrate the machine learning (ML) score distribution of the two cell populations. The classification method utilized a support vector machine (SVM). The ML score may also be referred to as an SVM score. The SVM obtained a receiver operating characteristic-area under the curve (ROC-AUC) of 0.993 for the sample depicted in FIG. 4B and a ROC-AUC of greater than 0.999 for the sample depicted in FIG. 10F.

### Example 3: Classification of STAT3 Cytoplasmic or Nuclear Localization

FIG. 4C shows fluorescence images of HepG2 cells comprising fluorescently tagged STAT3 proteins localized within a nucleus (nuclear) and outside of a nucleus (cytoplasmic). Cells were labeled using immunofluorescence (IF) against STAT3. Wild type (WT) HepG2 cells, with or without interleukin 6 (IL-6) stimulation, were utilized as a training set. Cells stained for STAT3 in the presence or absence of IL-6 were differentiated by visual analysis using fluorescence imaging. The right panel of FIG. 4C illustrates HepG2 cells stained for STAT3 in the absence of IL-6, and the left panel of FIG. 4C illustrates HepG2 cells stained for STAT3 in the presence of IL-6. IL-6 activates STAT3 and promotes localization of STAT3 to the nucleus, as seen by an accumulation of fluorescence near the cell center in the presence of IL-6.

For the screening set, the pooled sgRNA was transduced to Cas9-expressing HepG2 cells or the pooled peptide expression library was transduced to HepG2 cells. STAT3 was activated by IL-6 to screen for functional genes or peptides that inhibit nuclear translocation of STAT3. For the training set, WT HepG2 cells were trypsinized and centrifuged at low speed for 5 minutes. After removal of the supernatant, 1 mL of fresh RPMI 10% FBS medium was added and aliquot into two Eppendorf tubes. One was used for IL-6 stimulation and another for unstimulated control. For the STAT3 activation, HepG2 cells and the screening set were treated with 100 micrograms per milliliter (μg/mL) of IL-6 and incubated at 37° C. for 15 minutes. After centrifugation and removal of the supernatant, cells were fixed with 500 microliters (μL) 4% formaldehyde for 15 minutes at room temperature. Then, a few washes with PBS buffer were performed. After that, the cells were permeabilized with 500 μL of permeabilization buffer (Methanol, Acetone [1:2]), at 4° C. for 30 minutes. After washing with PBS, cells were blocked with 500 μL of 5% BSA (in PBS) and incubated at room temperature for 1 hour. Then, cells were washed with PBS and incubated with 125 μL primary STAT3 antibody (50×dilution in PBS) and incubated for overnight at 4° C. Then, cells were washed with PBS for a few times to remove non-specific binding and the pellets were re-suspended with 300 μL of secondary rabbit antibody (Goat anti-Rabbit IgG-488-100×dilution in PBS). After 60 minutes of incubation at room temperature (dark), the cells were washed by PBS three times and finally the pellet was re-suspended with 300 μL of PBS. Cell number was counted and adjusted to 1×10⁶ cells/mL.

FIG. 4D shows classification of HepG2 cells with nuclear-localized STAT3 (right peak, “IL6_plus”) and cells with cytoplasmic STAT3 (left peak, “IL6 minus”) proteins using systems and methods of the present disclosure. Cells were stained using immunofluorescence against STAT3. and cells treated with IL-6 or untreated cells were classified based on the localization of STAT3. The two peaks shown in each of FIG. 4D illustrate the SVM score distribution of the two cell populations based on the similarity to a phenotype of STAT3 localized to the nucleus. The images were classified using the imaging procedures disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference. The classification method utilized a SVM. The SVM obtained a ROC-AUC of 0.962.

### Example 4: Classification of NF-κB Cytoplasmic or Nuclear Localization

FIG. 10A shows fluorescence images of THP-1 cells comprising fluorescently tagged NF-κB proteins localized in the nucleus or the cytoplasm. Upon stimulation with lipopolysaccharide (LPS), subunits of NF-κB translocate to the nucleus. Cells were labeled using immunofluorescence against NF-κB. THP-1 cells, with or without LPS stimulation, were utilized as a training set. Cells stained for NF-κB in the presence or absence of LPS stimulation were differentiated by visual analysis using fluorescence imaging. The left panel of FIG. 10A illustrates THP-1 cells stained for NF-κB in the absence of LPS stimulation. The right panel of FIG. 10A illustrates THP-1 cells stained for NF-κB in the presence of LPS stimulation. Addition of LPS promotes translocation of NF-κB subunits to the nucleus.

FIG. 10B shows classification of THP-1 cells with nuclear localized NF-κB and cytoplasmic NF-κB using the systems and methods of the presence disclosure. Cells were stained using immunofluorescence against NF-κB, and cells pre-treated with LPS and untreated cells were classified based on nuclear localization (right peak, “LPS (+)”) or cytoplasmic localization (left peak, “LPS (−)”) of NF-κB. The two peaks shown in each of FIG. 10B illustrate the machine learning (ML) score distribution of the two cell populations based on the similarity to a phenotype with NF-κB nuclear localized. The images were classified using the imaging procedures disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference. The classification method utilized a SVM. The SVM obtained a ROC-AUC of 0.997.

FIG. 8A and FIG. 8B illustrate a method of training a machine learning algorithm to classify cells based on localization of a fluorescently tagged protein. A training set, shown in FIG. 8A, was generated using fluorescence images of THP-1 cells stained by immunofluorescence for the p65 subunit of NF-κB (“anti-p65”). Upon stimulation with LPS, p65 localizes to the nucleus. Cells were co-stained with a nuclear stain (e.g., DAPI or Hoechst). Labeled cells stimulated with LPS were used as a training set for cells positive for nuclear localization (FIG. 8A, right column), and unstimulated labeled cells were used as a training set for cells negative for nuclear localization (FIG. 8A, left column). Waveforms, as shown in FIG. 8B top panel, were generated from the stimulated and unstimulated data sets. The waveforms for each of the stimulated and unstimulated data sets were used to develop a machine learning model to classify cells positive for nuclear localization or negative for nuclear localization. The classification model utilized a SVM to discriminate between cells having nuclear localized NF-κB (right peak, “stimulated”) and cells having cytoplasmic NF-κB (left peak, “unstimulated”), as shown in the bottom panel of FIG. 8B. The trained model was able to distinguish between the waveforms of the stimulated and unstimulated cells.

### Example 5: Sorting of Cells Based on Cytoplasmic or Nuclear Localization of a Target Protein

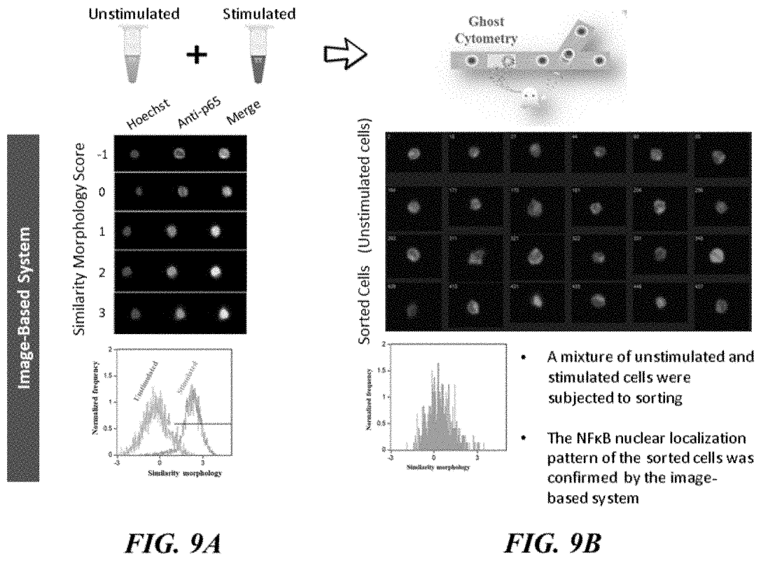

FIG. 9A and FIG. 9B show cells sorted based on cytoplasmic or nuclear localization of a target protein. Cells were stained by immunofluorescence for the p65 subunit of NF-κB (“anti-p65”). Stained cells were stimulated with LPS to promote nuclear localization of p65 and pooled with stained cells in the absence of stimulation (FIG. 9A, top panel). The pooled cells were either classified using an image-based system (FIG. 9A, middle and bottom panels) or sorted using ghost cytometry image-based cell sorting (FIG. 9B, top panel), as disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference. Ghost cytometry sorting was based on fluorescence intensity and morphology, as determined by previously obtained training sets. Training sets were obtained as described in Example 4 and Example 5. Cells were sorted into “stimulated” and “unstimulated” subpopulations based on the localization of the fluorescently tagged p65 protein. The subpopulation of cells classified as unstimulated was further subjected to analysis using an image-based system to confirm the sorting (FIG. 9B, middle and bottom panels). Prior to sorting, the pooled cells comprised two distinct cell populations (FIG. 9A). Following sorting, the selected cell subpopulation contained a single population (FIG. 9B), indicating that ghost cytometry was able to effectively sort cells based on the nuclear localization of fluorescently labeled p65.

### Example 6: Classification of Unaggregated and Aggregated Proteins Inside Cells

FIG. 4E shows fluorescence images of unaggregated or aggregated proteins inside HEK293T cells. Cells were stained using a Fluoppi system to detect protein-protein interactions between induced myeloid leukemia cell differentiation protein (MCL-1) and a Bcl-2 homologous antagonist/killer protein (BAK). The Fluoppi system facilitates the detection of protein-protein interactions by promoting the formation of fluorescent puncta or foci upon interaction between the target proteins. Cells stained with Fluoppi for MCL-1/BAK interactions, in the presence or absence of the MCL-1 inhibitor A-1210477, were used as a training set. A-1210477 inhibits MCL-1 and disrupts the interaction between MCL-1 and BAK. Cells stained for MCL-1/BAK interactions, in the presence or absence A-1210477, were differentiated by visual analysis using fluorescence imaging. The right panel of FIG. 4F illustrates HEK293T cells in the absence of A-1210477. In the absence of A-1210477, MCL-1 and BAK stained with the Fluoppi system form visible fluorescent foci. The left panel of FIG. 4F illustrates HEK293T cells in the presence of A-1210477. Addition of A-1210477 disrupts interactions between MCL-1 and BAK.

FIG. 4F shows classification of cells with unaggregated proteins or aggregated proteins using systems and methods of the present disclosure. The Fluoppi protein-protein interaction visualization system was used to detect protein interactions through the formation of detectable aggregates. Briefly, Fluoppi operates by affixing a multimeric fluorescent protein tag to a first labeled protein and an assembly helper tag to a second labeled protein. When the first and second labeled proteins interact, the interaction between the fluorescent protein tag and the assembly helper tag forms an optically detectable oligomeric protein complex. The complex may be detected as foci comprising protein aggregates or oligomers. Here, interactions between MCL-1 and BAK were probed using the Fluoppi system. Interactions between MCL-1 and BAK were disrupted using the MCL-1 inhibitor A-1210477. Cells were classified based on the presence of detectable protein aggregates (left peak, “positive”) or the absence of detectable protein aggregates (right peak, “negative”). The two peaks shown in each of FIG. 4F illustrate the SVM score distribution of the two cell populations based on the similarity to a phenotype of protein-protein interaction. The images were classified using the imaging procedures disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference. The classification method utilized an SVM.

### Example 7: Classification of a Protein-Protein Interaction Inside Cells

Ghost cytometry was implemented to acquire waveforms of fluorescence signals that contain image information (such as protein localization information) from both protein-protein interaction (PPI)-positive cells and PPI-negative cells. Then, each cell type was set as a training set to develop a machine learning model to classify PPI-positive cells and PPI-negative cells. The classification model utilized an SVM to discriminate between PPI-positive cells and PPI-negative cells.

FIG. 10C shows fluorescence images of CHO-K1 cells labeled for protein-protein interactions between p53 and mouse double minute 2 homolog (MDM2). Proteins p53 and MDM2 were labeled with the Fluoppi system, as described in Example 6. The left panel of FIG. 10C shows untreated cells, and the right panel of FIG. 10C shows cells treated with Nutlin-3. Nutlin-3 disrupts the interaction between p53 and MDM2, as seen by the dispersion of fluorescence upon treatment with Nutlin-3 as compared to the untreated sample. Cells labeled for protein-protein interactions between p53 and MDM2 and either treated with Nutlin-3 or untreated were used as a training set. Cells with or without Nutlin-3 were differentiated by visual analysis using fluorescence imaging.

FIG. 10D shows classification of cells positive (right peak, “untreated”) or negative (left peak, “Nutlin-3”) for protein-protein interactions between p53 and MDM2 using systems and methods of the present disclosure. Nutlin-3 was used to disrupt the interaction between p53 and MDM2. Cells were stained for interactions between p53 and MDM2, and cells treated with Nutlin-3 or untreated cells were classified based on interactions between p53 and MDM2. The two peaks shown in each of FIG. 10D illustrate the ML score distribution of the two cell populations based on the similarity to the phenotype of interactions between p53 and MDM2. The images were classified using the imaging procedures disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference. The classification method utilized a SVM. The SVM obtained a ROC-AUC of 0.958.

FIG. 4G shows fluorescence images of THP cells with nuclear-localized NF-κB proteins and cells with cytoplasmic NF-κB proteins. In order to identify an NF-κB nuclear translocation inhibitor, NF-κB localized in cytosol (FIG. 4G, right panel) and NF-κB stimulated by lipopolysaccharides (LPS) to induce nuclear translocation (FIG. 4G, left panel) were stained as a positive and negative training set respectively. LPS stimulation promotes dissociation of p65 from the NF-κB complex and translocation of p65 to the nucleus. Image information of the cells was acquired and classified using the imaging procedures disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096. which is entirely incorporated herein by reference. For a screening set, the pooled sgRNA library was transduced Cas9-expressing THP-1 cells or the pooled peptide expression library was transduced WT THP-1 cells. Upon stimulation with LPS, the classification model was applied to a screening set to sort the cells without NF-κB nuclear translocation. Plasmids from the sorted cells were isolated and subjected to next generation sequencing analysis to identify the genes or peptides that inhibit nuclear localization of NF-κB.

FIG. 4H shows classification of THP-1 cells based on nuclear-localized p65 subunit of NF-κB and the cells with cytoplasmic p65 subunit of NF-κB proteins using systems and methods of the present disclosure. LPS was used to stimulate localization of p65 to the nucleus. Cells were classified based morphology and localization of fluorescently labeled p65 in the presence (right peak, “stimulated”) or absence (left peak, “unstimulated”) of LPS stimulation. The images were classified using the imaging procedures disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference. The classification obtained a ROC-AUC of 0.972.

### Example 8: Validation of Rapid Image-Based Pooled Genetic/Peptide Screening (RIPGS)

FIG. 11 shows validation of a rapid image-based pooled genetic/peptide screening method using an image-based system and FACS. Cells were stained by immunofluorescence for the p65 subunit of NF-κB (“anti-p65”). Stained cells were stimulated with LPS to promote nuclear localization of p65. Stimulated cells were pooled with stained cells that were not subjected to LPS stimulation. Pooled cells were subjected to ghost cytometry sorting according to their fluorescence intensity together with the ghost signals obtained previously on the training sets. Training sets were obtained as described in Example 4 and Example 5. The sorted cells were collected, concentrated and checked for purity using an image-based system and a conventional FACS. Ghost cytometry image-based cell sorting (FIG. 11, top left panel) was performed as disclosed in S. Ota, R. Horisaki, Y. Kawamura, M. Ugawa, I. Sato, K. Hashimoto, R. Kamesawa, K. Setoyama, S. Yamaguchi, K. Fujiu, K. Waki, and H. Noji, “Ghost Cytometry”, Science 360(6394), pp. 1246-1251 (2018), doi: 10.1126/science.aan0096, which is entirely incorporated herein by reference. Cells were sorted into “stimulated” and “unstimulated” subpopulations based on the localization of the fluorescently tagged p65 protein. The subpopulation of cells classified as unstimulated was further subjected to FACS (FIG. 11, top center panel) or analysis using an image-based system to confirm the sorting (FIG. 11, top right panel and bottom panel). The ROC curve (Roc-AUC), and accuracy (Acc) obtained from the ghost cytometry sorting were 0.959 and 0.912, respectively, indicating a high degree of purity. The results were comparable with the ones calculated using FACS and the image-based system (purity of 83.6% and 86.7% respectively) and indicate that ghost cytometry was able to effectively sort cells based on the nuclear localization of fluorescently labeled p65.

### Example 9: Genetic Screening Based on Screening of Images

The systems and methods of the present disclosure may be used to perform rapid image-based pooled genetic and/or peptide screening for a variety of purposes. For instance, the systems and methods may be used to identify genes or peptides that alter fluorescence images (e.g. nuclear translocation, protein aggregation, and the like) of cells. Alternatively or in combination, the systems and methods may be used to identify genetic sequences that alter a structure and/or function of one or more peptides polypeptides, and/or proteins expressed by a cell.

The alterations may be detected by the imaging systems and methods described herein. For instance, the alterations may be detected based on whether or not an image displays proteins localization, protein-protein interactions, translocation, co-localization, puncta, or other attributes).

The systems and methods described herein may be utilized to identify one or more peptides, polypeptides, and/or proteins that block nuclear translocation of transcription factors, dissociate protein aggregation, restore organelle function, or the like.

The system and methods described herein may be utilized to identify undruggable targets, such as transcription factors, phosphatase RAS, or protein sites such as PPI sites. For instance, sites involved in PPI may often comprise relatively large, relatively flat surfaces that are generally not inhibited by small drug-like molecules. However, such sites may be inhibited by larger molecules such as peptides, polypeptides, and/or proteins. As another example, transcription factors may be flexible, lack ordered structure, or may be structurally heterogeneous. Transcription factors may adopt their proper three-dimensional structures only when within a cell. Thus, screening against transcription factors may require screening within a native cellular environment of the transcription factors. The methods described herein may be used to screen undruggable targets (e.g., transcription factors, phosphatase RAS, or protein sites) in their native cellular environment.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-45. (canceled)

46. A method for identifying a nucleic acid molecule, comprising:
(a) providing a cell that has been or is suspected of having been transfected or transduced with at least one exogenous ribonucleic acid (RNA) molecule or at least one exogenous deoxyribonucleic (DNA) molecule, wherein said cell is among a population of cells;
(b) subsequent to (a), identifying a morphological change of said cell;
(c) processing a content(s) of said cell to identify a nucleic acid molecule, a peptide, a polypeptide, or a protein;
(d) analyzing said nucleic acid molecule, said peptide, said polypeptide, or said protein identified in (c) to determine a presence of said at least one exogenous RNA molecule or at least one said exogenous DNA molecule; and
(e) using said presence of said at least one exogenous RNA molecule or said at least one exogenous DNA molecule to determine that said at least one exogenous RNA molecule or said at least one exogenous DNA molecule caused said morphological change of said cell.

47. The method of claim 46, wherein said morphological change comprises one or more members selected from the group consisting of: a change in a protein-protein interaction within said cell, a change in protein localization within said cell, a change in shape of said cell, a change in shape of one or more components of said cell, and a change in shape of one or more organelles of said cell.

48. The method of claim 46, wherein said cell is transfected using a plasmid comprising said at least one exogenous RNA molecule or said at least one exogenous DNA molecule.

49. The method of claim 46, wherein (c) comprises identifying a sequence of said nucleic acid molecule, said peptide, said polypeptide, or said protein from said cell.

50. The method of claim 46, wherein in (b), said morphological change of said cell is identified while said cell is flowing in a flow cell or flow channel.

51. The method of claim 46, wherein (b) is repeated for each cell of a plurality of cells among said population of cells.

52. The method of claim 51, wherein (b) is repeated at a rate of at least about 1500 cells per second (cells/s), at least about 2000 cells/s, at least about 3000 cells/s, at least about 4000 cells/s, at least about 5000 cells/s, at least about 6000 cells/s, at least about 7000 cells/s, at least about 8000 cells/s, at least about 9000 cells/s, or at least about 10,000 cells/s.

53. The method of claim 46, wherein said cell is isolated from said population of cells based on said morphological change.

54. The method of claim 53, wherein each cell among said population of cells comprises at least one randomly-inserted exogenous RNA molecule or at least one randomly-inserted exogenous DNA molecule.

55. The method of claim 46, wherein (b) comprises imaging said cell.

56. The method of claim 46, wherein (b) comprises obtaining temporal signals containing (i) image information or (ii) spatial information of said cell.

57. The method of claim 56, wherein said temporal signals are transformed into (i) time-independent image information or (ii) time-independent spatial information of said cell.

58. The method of claim 46, further comprising using at least said morphological change to determine that said at least one exogenous DNA molecule or said at least one exogeneous RNA molecule inhibits or activates a biochemical pathway within said cell.

59. The method of claim 46, wherein (b) comprises using a machine learning algorithm to identify said morphological change.

60. The method of claim 59, wherein said machine learning algorithm comprises one or more members selected from the group consisting of: support vector machines, random forest, artificial neural networks, convolutional neural networks, deep learning, ultra-deep learning, gradient boosting, AdaBoosting, decision trees, linear regression, and logistic regression.

61. The method of claim 59, wherein said machine learning algorithm is trained using a training set comprising image information of a cell population positive for said morphological change and image information of a cell population negative for said morphological change.

62. The method of claim 61, wherein said image information comprises temporal signals containing (i) time-independent image information or (ii) time-independent spatial information of said cell.

63. The method of claim 59, further comprising, prior to (b), labeling (1) at least of one of said peptide, said polypeptide, or said protein within said cell or (2) at least one of another peptide, another polypeptide, or another protein within another cell among said populations of cells.

64. The method of claim 63, wherein said labeling comprises labeling said peptide, said polypeptide, or said protein in (1) or (2) with one or more fluorescent labels, Forster resonance energy transfer (FRET) labels, dyes, fluorophores, or quantum dots.

65. A system for identifying a nucleic acid molecule, comprising one or more computer processors that are individually or collectively programmed to:
(a) identify a morphological change of a cell, which cell has been or is suspected of having been transfected or transduced with at least one exogenous ribonucleic acid (RNA) molecule or at least one exogenous deoxyribonucleic (DNA) molecule, wherein said cell is among a population of cells;
(b) process a content(s) of said cell to identify a nucleic acid molecule, a peptide, polypeptide, or a protein;
(c) analyze said nucleic acid molecule, said peptide, said polypeptide, or said protein identified in (c) to determine a presence of said at least one exogenous RNA molecule or said at least one exogenous DNA molecule; and
(d) using said presence of said at least one exogenous RNA molecule or said at least one exogenous DNA molecule to determine that said at least one exogenous RNA molecule or said at least one exogenous DNA molecule caused said morphological change of said cell.
